(19) United States
(12) Patent Application Publication
(10) Pub. No.: US 2025/0328030 A1
(43) Pub. Date: Oct. 23, 2025

(54) POPULATION BASED EYEWEAR FITTING

(71) Applicant: Materialise N.V., Leuven (BE)

(72) Inventors: Tom CLUCKERS, Leuven (BE); Philippe LAES, Leuven (BE); Jan MAES, Leuven (BE); Alireza PARANDIAN, Leuven (BE)

(21) Appl. No.: 19/216,565

(22) Filed: May 22, 2025

Related U.S. Application Data
(63) Continuation of application No. PCT/US2023/081206, filed on Nov. 27, 2023.
(60) Provisional application No. 63/428,383, filed on Nov. 28, 2022.

(51) Int. Cl. G02C 7/02; A61B 5/107; B33Y 50/00; B33Y 80/00

(52) U.S. Cl. CPC G02C 7/027; A61B 5/1077; B33Y 50/00; B33Y 80/00

(57) ABSTRACT

Certain aspects provide a method for creating a discrete set of variants of a customizable wearable object, comprising: receiving a baseline 3D design file for a baseline 3D customizable wearable object; obtaining a fitting population comprising a set of 3D anatomy models representing potential wearers of the customizable wearable object; generating a plurality of modified 3D customizable wearable objects based on fitting the baseline 3D customizable wearable object to each 3D anatomy model of the set of 3D anatomy models; selecting one or more of the plurality of modified 3D customizable wearable objects as the discrete set of variants of the customizable wearable object; and generating a set of 3D design files corresponding to the discrete set of variants of the customizable wearable object.

### CROSS-REFERENCE TO RELATED APPLICATIONS

This Application is a continuation of International Application No. PCT/US2023/081206, filed on Nov. 27, 2023, which claims the benefit of and priority to U.S. Provisional Patent Application No. 63/428,383, filed on Nov. 28, 2022, the entire disclosures of both of which are hereby incorporated by reference.

### BACKGROUND

### Field of the Invention

Certain disclosed embodiments relate generally to techniques for creating a set of different fits of a wearable product design. Different fits of a wearable product design may refer to versions of the wearable product design that have different sizes, ratios of dimensions between components, relative position of components, and/or relative angles of components, but the fits still share the same design aesthetic.

An example of a wearable product is an eyewear product, such as a pair of prescription glasses, VR glasses, AR glasses, XR glasses, sunglasses, etc. Another example of a wearable product is a wristwatch, such as a smart watch. Another example of a wearable product is a footwear insole.

### Description of the Related Technology

Traditionally many wearable products, such as eyewear products, are mass-produced, with only one size of a particular design available to the end-user consumers, without the option of choosing a product that is a good fit for their personal anatomy.

For example in eyewear applications, conventional eyewear configuration begins with an eyewear frame with a fixed model and size. Once an end-user in an optician's shop has chosen a preferred frame, the optical lenses that fit the end-user's visual needs are selected and placed into the frame. The drawback of this approach is that, without any alteration to the frame, there is a risk that the frame does not allow sufficient alignment of the lens with the eyes of the end-user to ensure ideal performance of the eyewear product. Other drawbacks of a chosen eyewear frame that is not the right fit, are that it may not be aesthetically pleasing to the end-user, and/or that it may not be comfortable to wear. In case of smart eyewear, configuration of the glasses requires positioning of the optical components in front of the eyes, with each pupil aligned within the eyebox. With an eyewear frame that is not the right fit, there is a risk that the projected displays will not be fully visible to the end-user.

It is possible to provide an end-user with randomly chosen pre-determined multiple sizes for a given eyewear frame, from which the most suitable frame can be chosen to increase alignment of the inserted optical lenses with the end-user's eyes. The drawback of this approach is that it is difficult to determine the suitable sizes that will accommodate a large number of end-users. Another drawback is that a large number of frames would have to be produced and kept in stock to allow end-users to try the different frames and decide on the most suitable size.

Fully customized eyewear configuration uses 3D scanning, parametric design automation and 3D printing to design and manufacture the frame of an end-user's choice. An eyewear product that is fully customized to the facial anatomy of the end-user will guarantee optimal alignment of the lens with the eyes of the end-user, ensuring ideal performance of the eyewear product, both functionally and aesthetically. The use of 3D printing permits efficient manufacturing of the one-off fully customized eyewear product. Every frame that is produced is entirely unique and adapted to an individual end-user. The drawback of this approach is that it is time-consuming to set up a working 3D-printing solution for fully customized eyewear frames. The frames are manufactured, for example using laser sintering technology tuned to highly precise parameters, and once manufactured undergo a multi-stage post-production treatment.

It should be noted that the information included in the Background section herein is simply meant to provide a reference for the discussion of certain embodiments in the Detailed Description. None of the information included in this Background should be considered as an admission of prior art.

### SUMMARY

In one embodiment, a computing device is provided for creating and/or selecting customized wearable objects (also referred to as products or wearable products). The computing device may include a memory configured to store a baseline 3D design file (e.g., CAD file, STL file, etc.) for an object, customization data for the object, and/or product information associated with the object. The customization data may be aimed at ensuring that the wearable object is not modified to a degree that it no longer meets the intended functional and/or aesthetic purpose, that it deviates too strongly from the original design and proportions, and/or that it can no longer be manufactured, for example by 3D printing.

The computing device may further include one or more processors (e.g. for application and visualization services) in data communication with the memory (e.g. for design storage). The one or more processors may be configured to execute one or more computer-readable instructions, such as stored in the memory, to cause the computing device to create and/or select customized wearable objects. The instructions may be written as one or more services or modules, as further discussed herein. A service or module may include code to process data, and may access memory or storage to store data. However, it should be noted that one of ordinary skill in the art will understand that the instructions need not be written as separate services or modules. Further, where services or modules are described as storing data, this may refer to the data being stored in a suitable storage and/or memory accessible by the processor executing the instructions corresponding to the described service or module. For example, an application services module may include a population-based sizing module comprising population data including stored actual 3D anatomy models representing a base population of users. Each actual 3D anatomy model (also referred to as a measured 3D anatomy model) may include a 3D model and/or measurement information corresponding to an anatomy of an individual, such as derived from 3D scans of the anatomy of the individual. In certain aspects, the population-based sizing module may be configured to generate additional virtual 3D anatomy models (also referred to as generated 3D anatomy models), which may be linear combinations (or some other derivative) of the actual 3D anatomy models (e.g. 3D anatomy scans) included in the population data. Accordingly, the techniques described herein may be performed on a set of 3D anatomy models, which may include actual and/or virtual 3D anatomy models. The set of 3D anatomy models may be referred to as a fitting population, including a base population corresponding to the actual 3D anatomy models and a virtual population corresponding to the virtual 3D anatomy models. For example, a base population may include actual 3D anatomy models corresponding to 3D anatomy scans of individuals, and a virtual population may include virtual 3D anatomy models, such as corresponding to linear combinations of the actual 3D anatomy models of the base population.

The services may include a service for storing fitting data. The stored fitting data may be indicative of design modifications made to at least one baseline 3D design so as to fit it to a 3D anatomy model. The application services module may further include a full customization module configured to virtually superimpose the baseline 3D design over a 3D anatomy model, and to modify the baseline 3D design file for the object to fit the anatomy in the 3D anatomy model, such as based on the customization data and the fitting data. The full customization module may be configured to make changes to a 3D anatomy model. For example, in eyewear applications, in order to ensure centration of the pupils of a wearer within an eyewear frame, the location of the pupils may be indicated on the 3D anatomy model as specific reference points (landmark points).

The population-based sizing module may further be configured to receive instructions generated by the full customization module, and to perform an initial batch fitting on the fitting population, resulting in a set of customized wearable object files comprising modified versions of the baseline 3D design file, the modified versions being customized to the anatomies represented in the 3D anatomy models of the fitting population, such as according to customization data and fitting data. The customized wearable object files may correspond to a set of virtual 3D objects (e.g., set of eyeglasses). The population-based sizing module may further be configured to determine or generate a set of 3D files storing a discrete set of variants (i.e., a limited number of variants) of the versions (e.g., modified and/or unmodified) of the baseline 3D design of the wearable object, the discrete set of variants accommodating a certain portion of the 3D anatomy models of the fitting population, such as when taking into account customization data and fitting data. In certain aspects, to generate the discrete set of variants, the population-based sizing module is configured to minimize a number of variants and maximize a number of 3D anatomy models to which the number of variants fit, such as using a suitable clustering algorithm, or machine learning algorithm. In certain aspects, the population-based sizing module is configured to cluster the set of customized wearable object files into groups, each group including similar customized wearable object files in that the customized wearable object files in the group fit on similar or even the same 3D anatomy models. In certain aspects, the population-based sizing module is configured to select a representative customized wearable object file for each group, such as so that the representative customized wearable object file fits all the 3D anatomy models used to generate the customized wearable object files of the group.

The applications services module may further include an end-user fitting module configured to receive scanning data and user specifications from an end-user/purchaser of a wearable object. The end-user fitting module may further be configured to receive from the full customization module, an end-user customized 3D design file that includes a fully customized 3D design of the wearable object that fits the 3D scan of the end-user, such as taking into account the received user specifications, the stored customization data, and/or the stored fitting data. The end-user fitting module may further include a comparison metric and may further be configured to allocate a fit score to rate the overlap of the end-user customized design file and each of the 3D files of the discrete set of variants. The allocation of the fit score may take into account the fitting data and the user specifications. The end-user fitting module may further be configured to select one or more of the discrete variants of the 3D baseline design for the individual end-user of the wearable object based on the fit score.

The system may further include visualization services in data communication with the application services, and configured to generate a visualization or display of the selected discrete variant superimposed over the 3D anatomy scan of the end-user. The visualization services may further be configured to display fitting features, providing visual markers with which to align the variant and the 3D anatomy scan. For example, in eyewear applications, the displayed fitting features may include the visualization of the position of an end-user's pupils compared to an eyewear frame, the fitting feature ensuring alignment of a wearer's pupil and the eyewear frame lens. The position of the pupil with respect to the lens may be visualized in combination with measurements, such as cornea vertex distance, distance between lenses, eyepoint height, distance between lower frame rim and pupil, distance between upper frame rim and pupil, and pantoscopic angle. Another example of a displayed fitting feature in eyewear applications is the visualization of the alignment of an end-user's pupil within the eyebox of a pair of smart glasses.

The system may be further configured to transmit the selected discrete variant to a manufacturing service for manufacturing of the object, for example by additive manufacturing techniques such as three-dimensional printing, or by milling.

In another embodiment, a method is provided of creating a set of discrete variants of an object and/or selecting a variant for a specific end-user. The method may include receiving data indicative of a baseline 3D design for an object, customization data for the object, and/or product information associated with the object. The method may further include receiving fitting data for the object and population data comprising a set of 3D anatomy scans representing a particular base population of wearers of the object. The method may further include receiving a 3D scan image associated with an anatomical feature of an individual end-user/purchaser of the object and/or user specifications provided by the end-user/purchaser.

The method also may include generating a virtual population of 3D anatomy models, such as based on linear combinations, from the 3D anatomy scans of the base population. The method also may include modifying the baseline design to fit each of the 3D anatomy models of a fitting population, such as according to the received customization data and/or the received fitting data, resulting in a set of virtual 3D objects. The method also may include generating or selecting a representative set of variants of the virtual 3D objects from the set of virtual 3D objects, which may corresponding to the discrete set of variants discussed herein. For example, selecting the representative set of variants of the virtual 3D objects may include clustering the virtual 3D objects into a discrete number of groups and identifying a representative virtual 3D object/design for each cluster, the representative virtual 3D objects/designs corresponding to a discrete set of variants of the virtual 3D objects. The method also may include modifying the baseline 3D design to fit the 3D scan of the individual end-user/purchaser, such as according to the received customization data, the received fitting data, and/or the received user specifications, resulting in an end-user customized 3D object. The method also may include comparing each discrete variant of the 3D object to the end-user customized 3D object and based on a comparison metric allocating a fit score to each discrete variant. The method may further include selecting the discrete variant with a suitable (e.g., threshold, highest, etc.) fit score for the individual end-user of the wearable object.

The method also may include generating a visualization or display of the selected discrete variant superimposed over the 3D anatomy scan of the end-user.

The method may also include transmitting the selected discrete variant to a manufacturing service for manufacturing of the object, for example by additive manufacturing techniques such as three-dimensional printing, or by milling.

In another embodiment, a non-transitory computer readable medium comprising computer-executable instructions is provided. When the computer-executable instructions are executed by a processor, they may cause a computing device to perform a method of generating customized object files, selecting best fits of objects for end-users among sets of variants, and/or manufacturing selected objects, as described herein.

The above concepts may be executed in a single computing device, or alternatively across multiple computing devices, such as multiple networked devices.

### BRIEF DESCRIPTION OF THE DRAWINGS

FIG. 1 is a block diagram providing an example of a computer network environment suitable for implementing various embodiments described herein.

FIG. 2 is a block diagram of a computing device which may be used to performance various aspects of the embodiments described herein.

FIG. 3 is a block diagram showing an example of a scanning device which may be used in accordance with one or more embodiments.

FIG. 4 is a more detailed view of the design storage shown in FIG. 1.

FIG. 5a provides example customizable features for a pair of eyeglasses. FIG. 5b provides example customizable features for a wristwatch. FIG. 5c provides example customizable features for an insole.

FIG. 6 is a more detailed view of the application services from FIG. 1.

FIG. 7 provides an example of 3D facial models generated using a statistical shape model.

FIGS. 8a and 8b provide an example of clustering for 2 dimensions comprising customization parameters for an eyewear product. FIG. 8a shows a number of datapoints generated by a first batch fitting. FIG. 8b shows a number of datapoints, a number of clusters and a representative datapoint per cluster.

FIG. 9 is a flow chart showing a process by which a customized 3D product design can be created in accordance with one or more embodiments disclosed herein.

FIG. 10 is a flow chart showing a process by which a set of variants of a 3D product design can be created in accordance with one or more embodiments disclosed herein.

FIG. 11 is an example of an environment suitable for creating a three-dimensional scan of a person in order to customize a 3D printed object to their personal characteristics.

FIG. 12a is a flow chart showing a process by which a variant of a 3D product design is selected for a user in accordance with one or more embodiments disclosed herein. FIG. 12b is a flow chart showing an alternative process by which a variant of a 3D product design is selected for a user in accordance with one or more embodiments disclosed herein.

FIG. 13 is a flow chart showing a process where variants of a 3D product design are created in accordance with one or more embodiments disclosed herein.

### DETAILED DESCRIPTION

Working with a limited, discrete number of different sizes or fits of a wearable product, rather than working with a continuous range of fully customized models, reduces supply chain complexities during set-up and on-boarding of the manufacturing process. Onboarding and setting up a manufacturing system for fully customizable models, in which the dimensions of a wide range of parts can be altered, generates an infinite number of models, some of which may not meet aesthetic standards, or may not be manufacturable. It is more efficient to ensure a discrete number of models, for example between 5 and 10, meet aesthetic standards, and are manufacturable, for example by 3D printing.

Another advantage of working with a limited number of discrete models, rather than with fully customized models, is that it allows pushing forward of the customer order decoupling point. The customer order decoupling point defines the place in the supply chain for a product, where product is linked to a specific order. Processes upstream of the customer order decoupling point are driven by forecast-based information. Optimization is achieved by balancing inventory and capacity. Downstream processes are driven by concrete customer orders. Optimization is achieved by balancing capacity and process lead-times. A fully customized approach has a very early customer order decoupling point, a discrete model approach has a later customer order decoupling point. A later customer order decoupling point allows the build-up of stock. Having a view on which sizes are most common in a given population can provide statistical input on advisable stock for each individual discrete size.

A further advantage of working with discrete models, rather than with fully customized models is that mass-produced parts can be integrated into the product. For example, standard metal temples can be integrated into discrete models of eyewear frames of which the rest is 3D printed in a plastic material. The discrete models can be configured in such a way that the standard mass-produced parts can be integrated into the overall model.

WO2015/166048-A1 (Materialise) describes the customization of an object by generating a display superimposing a baseline 3D design of the object over a 3D scan of the anatomy of the end-user of the object, modifying the baseline design of the object according to customization parameters to ensure a customized fit to the anatomy of the scanned end-user, and 3D printing the customized object. This reference is incorporated herein in its entirety.

WO2021/239539-A1 (Zeiss) describes the selection of an eyewear frame using head data clusters based on the head data of the person, frame data clusters based on the identified head data cluster and a mapping between the plurality of head data clusters and the plurality of frame data clusters. It does not provide the possibility of predetermining a set of discrete size/fit models, each discrete model being based on a clusters of fully customized designs fit on a fitting population.

U.S. Pat. No. 9,254,081-B2 (Ditto) describes the use of a fit score allocated based on the fit of an eyewear frame for an end-user based on the comparison of end-user head measurements to a set of eyewear frame measurements. It does not provide the possibility of allocating a fit score based on a comparison of a range of discrete size models that accommodate a significant part of a particular population of end-users, to a fully customized product modified to fit a particular end-user.

System and methods for determining different variants of a baseline 3D design of a wearable object are provided. Embodiments of this application relate to systems and methods which allow for wearable objects, such as eyeglasses, wristwatches, and/or insoles, for example, to be customized to accommodate a broad range of users. The extent of the customization may be constrained according to specifications for the modification of the geometry and size of particular parts of the wearable objects. These customization constraints may be defined by manufacturers, designers, retailers and/or sellers of the wearable objects. The modification specifications may be constrained based on factors relating to the manufacturability of a modified object design. Modifying part of the wearable object may make the object difficult to manufacture, for example by 3D printing or by milling. A manufacturer may, for example, limit the reduction in size of a certain part of the wearable object because a part smaller than a certain threshold would be fragile which would make it prone to breaking during manufacturing. The modification specifications may also be constrained based on factors relating to the aesthetic requirements of the wearable object. Modifying part of the wearable object may make the object aesthetically unappealing. A designer may, for example, limit the changes in the proportions between the different parts of the wearable object, to avoid having a customized wearable object that no longer corresponds to the intended aesthetic of the original design. The modification specifications may also be constrained based on factors relating to the functional requirements of the wearable object. Modifying part of the wearable object may make the object less efficient in its intended use. A retailer/supplier may, for example in an eyewear application, limit the reduction of the lens contour to avoid difficulties in correctly positioning the eyewear lens in the frame for alignment of the lens center and the wearer's pupil. In some embodiments, specific zones may be defined on the baseline 3D design of the wearable object which are eligible for customization. These zones and their associated customization constraints may be interrelated. Based on their interrelationships, the modification constraints of the various zones may update in response to modifications made to other zones.

To facilitate supply chain management, a manufacturer or retailer/supplier of wearable objects may want to limit the number of customized products to a discrete set of variants of the original design. The geometries of the variant designs may be based on population data, providing a broad range of potential customers with a variant design that has geometries close to those of a fully customized design. The selection of the variant that most closely resembles a fully customized design may be based on additional user specifications supplied by the customer, such as personal preferences or for example for an eyewear product, lens prescription requirements. Thus, embodiments disclosed herein allow designers, manufacturers and/or retailers/suppliers to offer customers the ability to purchase a customized product taking into account the customer's preferences, requirements and/or physical characteristics, while at the same time maintaining sufficient control over the design as a whole so that the overall aesthetic qualities and functionality of the devices are not harmed.

For the purposes of this description:
“3D anatomy model” may refer to a 3D model (e.g., point cloud, CAD file, other 3D model) and/or measurement information (e.g., anatomy measurements such as measurements of one or more body features, such as distance between pupils, ear size, head width and height, etc.) corresponding to an anatomy.
“Actual 3D anatomy model” or “measure 3D anatomy model” may refer to a 3D anatomy model derived from 3D scans or measurements of an actual individual.
“Virtual 3D anatomy model” or “generated 3D anatomy model” may refer to a 3D anatomy model derived or generated from information, such as from one or more actual 3D anatomy models, such as based on linear combinations, machine learning techniques, interpolation, etc.
“Base population” may refer to a set of actual 3D anatomy models.
“Virtual population” may refer to a set of virtual 3D anatomy models.
“Fitting population” may refer a set of one or more actual 3D anatomy models and/or one or more virtual 3D anatomy models. For example, a fitting population may refer to a collection of one or more 3D anatomy models. The fitting population may include one or both of models produced from scans or measurements of actual people and models derived from (e.g., corresponding to a statistical distribution of) anatomy models, such as using linear combination or other interpolation techniques. The fitting population may be a digital representation of a wide variety of potential customers, allowing designers to see how their products will look and fit on a many different people.
“Baseline 3D design file for an object” may refer to an unmodified baseline design of an object (e.g., glasses, insole, footwear, eyewear, etc.), for example, having unmodified dimensions or other characteristics, which may be referred to as a “baseline 3D object.”
“Modified 3D design file for an object” may refer to a modified baseline design of an object, for example, having modified dimensions or other characteristics, which may be referred to as a “modified 3D object.” In certain aspects, modifications are constrained or limited by one or more of customization data, fitting data, and/or user specifications.
“Variants” or “discrete variants” of a baseline 3D design file for an object, or of the object itself, may refer to one or more modified 3D design files for the object, or one or more modified 3D objects, and may include the baseline 3D design file for the object itself, or the baseline 3D object itself.
“Customization data” may refer to information specifying a range of permissible object designs, such as a range of modifications that may be made to one or more spatial parameters (e.g., dimensions, angles, positions of portions, etc.) of a baseline 3D object or baseline 3D design file for an object. This information may include lists of customizable portions of the object, minimum and/or maximum sizes for one or more portions of the object, specific relationships (e.g., ratios or ranges of ratios) to be maintained between different portions of the object, acceptable angles of connections between portions of the object, or any other information necessary to define the range of permissible designs or modifications of the object. Customization data may include “customization constraints” and “manufacturing constraints” (or “printability constraints”). Customization constraints include functionality requirements and aesthetic requirements, ensuring the design functions as intended and matches the intended aesthetic. Manufacturing constraints ensure that the design is able to be successfully manufactured.
“Fitting data” (or “fitting methods”, “fitting algorithms”, or “fitting criteria”) may refer to information defining how the wearable object fits to the anatomy of a user. Fitting data may include a method for determining a suitable (e.g., optimal) customization for provided anatomy information (e.g., measurements) of a user, as well as a method for evaluating the fit of a provided object design (e.g., baseline or modified 3D design file for an object) to provided anatomy information. Fitting data may include how specific parts of the object should be sized based on particular features of the customer's anatomy, how much variation from the optimal design will still fit a customer, and/or thresholds where a design completely fails to fit. For example, as further discussed herein, fitting data may include fitting parameters and fitting constraints. In certain aspects, the parameters and their related constraints are taken into account when modifying a 3D customizable wearable object in order to fit a specific 3D anatomy model, such as so that the wearable object still performs as intended, keeps the same design language, and still remains comfortable to be worn by the user.
“User specifications” may refer to customization preferences particular to a specific customer. User specifications may additionally or alternatively include user preferences, such as color selection, a desire for an oversized product, or a need for particular materials. User specifications may additionally or alternatively include specification data from similar products, allowing design reuse. User specifications may include, for example, prescription data for eyewear products.
“Scanning data” may refer to measurements, image data, point data, etc., obtained for an anatomy of a user.
“Product information” may refer to include general sales information about products available for purchase from manufacturers and/or designers, product name, SKU, and/or descriptive information regarding the customizations that may be made to the product, such as explanatory information with regard to the customization data.

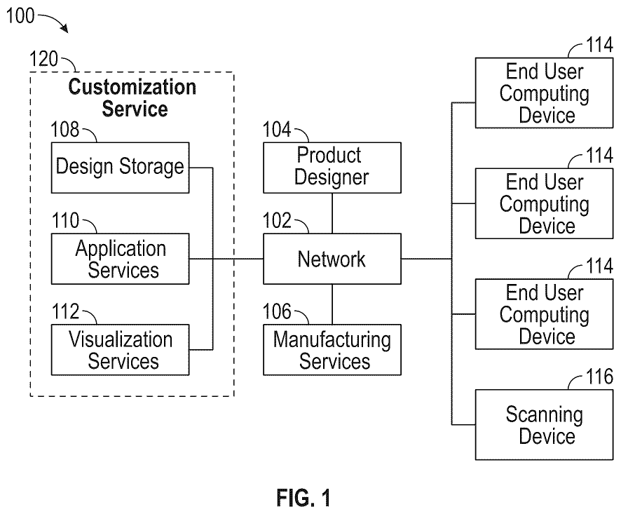

FIG. 1 shows an example of a computer network environment 100 suitable for implementing various embodiments. The network environment 100 includes a computer network 102. The computer network 102 may be any of various types and combinations of public and/or private networks. In some embodiments, the computer network 102 may be the Internet. In other embodiments, the computer network 102 may be a combination of the Internet and one or more private computer networks which are in data communication with the Internet via telecommunications routing equipment or some other means. In still other embodiments, the computer network may be a purely private network which uses proprietary protocols to transmit and receive data between various network devices.

The computer network environment 100 may further include an object/product design platform 104. The product design platform 104, typically associated with a product designer and/or manufacturer and/or retailer/seller, provides a computing environment which allows a product designer and/or manufacturer to create three-dimensional designs for their products. Those designs may be stored in a format suitable for generating the designed product, for example using additive manufacturing techniques such as three-dimensional printing. In some embodiments, the designs may be stored in a 3D printable STL file format. However, other suitable 3D print formats may be used.

The computer network environment 100 may also include manufacturing services 106, for example additive manufacturing services, or milling services. The manufacturing services 106 may be in data communication with the computer network 102. The manufacturing services may include advanced 3D printing technology, which enables the manufacture of a product based on a 3D printable file. In some implementations, the manufacturing services may be provided by the owner of the product design platform 104. Alternatively, the manufacturing services may be provided by a 3D printer associated with a consumer. In still other embodiments, the manufacturing services 106 may be provided by an additive manufacturing service provider that specializes in providing those services to customers.

The computer network environment 100 may also include a customization service 120. The customization service 120 may generally take the form of one or more computer systems which provide customization services for products designed via the product design platform 104. In some embodiments, the customization service 120 may include design storage 108. The design storage 108 may include a memory and/or storage in which designers may place designs. The design storage 108 may take the form of a network connected database which stores files, for example STL files and other 3D printable file formats.

The customization service 120 may also include application services 110. The application services 110 may take the form of one or more applications running on an application server which are configured to allow access to design data stored in the design storage 108.

The customization service 120 may further include visualization services 112. The visualization services 112 may take the form of a digital display on a traditional personal computing device, a mobile telephone device or a tablet computer, set-top box computer, or some other computer platform, which is in data communication with one or more of the application services 110 and the design storage 108. Alternatively, the visualization services may take the form of a Web server. In some embodiments, the visualization services 112 may be configured to provide browser-based access to the application services and design data provided within customization service 120. In some embodiments, the visualization services 112 may utilize off-the-shelf (“OTS”) software components. Alternatively, the visualization services 112 may be provided through a customized and/or proprietary web interface.

The computer network environment 100 may also include one or more end-user computing devices 114. The end-user computing devices 114 are typically associated with end-users, customers and/or consumers who are considering purchases of products designed or sold by the designer and/or manufacturer 104, or alternatively they may be associated with a retailer/seller. The end-user computing devices 114 may take various forms. In some embodiments, the end-user computing devices may be traditional personal computing devices running operating system such as Windows®, Linux, chrome OS, or Mac OS. The end-user computing devices 114 may also take the form of mobile telephone devices running mobile operating systems such as iOS, Android, or the like. The end-user computing devices 114 may also take the form of tablet computers, set-top box computers, or some other computer platform which can be used by an end user to connect to the computer network 102.

Some embodiments are able to customize manufactured objects to fit specific physical characteristics or attributes of an end-user. To that end, the computer network environment 100 may also include a scanning device 116. The scanning device typically takes the form of a 3D scanner which uses one or more cameras to develop a 3D image of a scanned person, object or a part thereof.

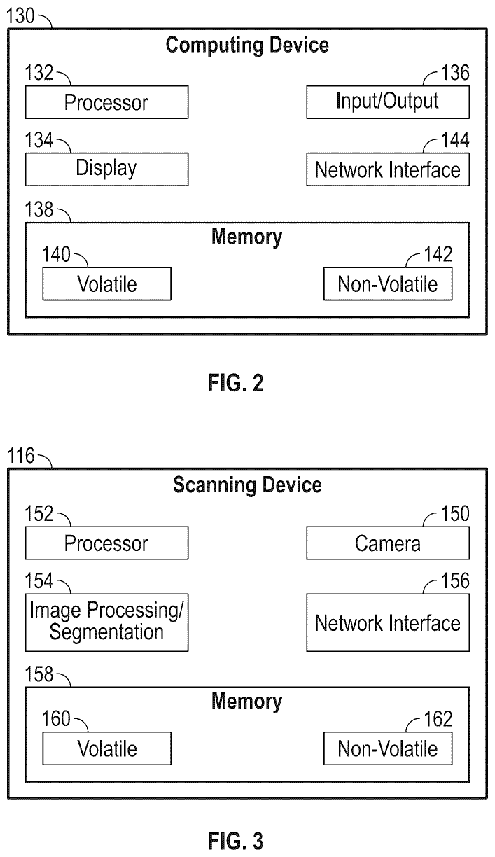

FIG. 2 shows an example of a computing device 130 that is suitable for implementing various aspects of the methods and techniques discussed herein. As noted above, end-user computing devices 114 may be of the various forms described. Other computers (as well as the end-user computing devices 114) present in the computer network environment 100 may also take the form of a computing device, such as computing device 130. The computing device 130 includes one or more processors, shown as a processor 132. The processor 132 may be a central processing unit (“CPU”), a graphics processing unit (“GPU”), and/or it may be a multipurpose processing unit such as a system on a chip (“SOC”) which provides both CPU services and other ancillary processing such as graphics, integrated network, or other features.

The computing device 130 may also include a display 134. The display 134 may take various forms. In some embodiments, the display is integrated into the computing device 130. Alternatively, the display 134 may be a separate display (or multiple displays) configured to output information to a graphical user interface. The computing device 130 may further include an input/output system 136. The input/output system 136 typically includes various input devices which allow a user to interact with the computing device 130. The input devices may include a mouse, a keyboard, a touchscreen, a microphone, and the like. The input/output system 136 also typically includes output components. The output components may be the display 134, some sort of tactile feedback mechanism, an audio output device such as a speaker, or some other form of output device.

The computing device 130 may also include one or more memories, shown as memory 138. The memory 138 is generally used to store information used in connection with the systems and methods described herein. The memory 138 may include volatile memory 140 such as some form of random access memory (“RAM”). The memory 138 may also include nonvolatile memory 142 which provides persistent storage of data. The nonvolatile memory 142 may take several forms. It may take the form of one or more hard disk drives, flash memory, read-only memory, optical disk, or some other form.

The computing device 130 may also include a network interface 144. The network interface 144 is typically a computer network interface card which provides access to the computer network 102 via any appropriate computer networking protocol. The network interface 144 may be a separate component of the computing device 130, or it may alternatively be part of the processing component 132. The network interface 144 may be a wired network interface, or maybe a wireless network interface.

It should be noted that though certain aspects of services and modules are described as being performed on certain computing devices, or over networks, etc., the various processes discussed herein may be performed on a single computing device or any suitable number of computing devices. Further, data may be stored locally on said single or multiple computing devices, or be accessible externally. Further, where aspects are described as performed by a processor and/or memory, the aspects may be performed by one or more processors and/or one or more memories.

FIG. 3 is a block diagram providing an example of various components that may be included in a scanning device 116 in accordance with various embodiments described herein. In general, the scanning device may be used to acquire the 3D shape of a target person, object or part thereof. The scanning device 116 may be a commercially available scanning device such as a 3DMD scanner, a GOM scanner, or a custom-built scanner. Alternatively, the scanning device 116 may be a specialized device which is designed to be fit for purpose. The scanning device 116 may implement any one of various 3D scanning techniques to obtain 3D scans of objects. These techniques may include contact-scanning. Alternatively, light-based 3D scanners may also be used. In the examples described herein, the scanning device 116 utilizes passive scanning techniques.

A scanning device 116 may include a camera system 150. The camera system 150 may include a single camera which is maneuverable to acquire images from various perspectives. Alternatively, the camera system may include a plurality of cameras positioned at various angles and perspectives with respect to a target area for scanning. The camera system may consist of a video camera system, for example a single or multiple camera system consisting of a handheld camera including a calibration device, an RGB image and depth sensor. The captured or filmed images obtained by the camera device 150 may be stored in memory 158. As was the case with the computing device 130, the memory may include volatile memory 160 and/or nonvolatile memory 162. The scanning device 160 may also include a processor 152. As with the computing device 130 above, the processor 152 may be a standard CPU unit, or it may be a system-on-a-chip unit. In still other implementations, the processor 152 may include one or more specialized processing units which are designed for processing imaging data and driving the scanning device. The scanning device 116 may also include an image processing module 154 and a network interface 156. The image processing module 154 is typically configured to receive the images from the camera and process them in order to create a data set that can be converted into a 3D design format.

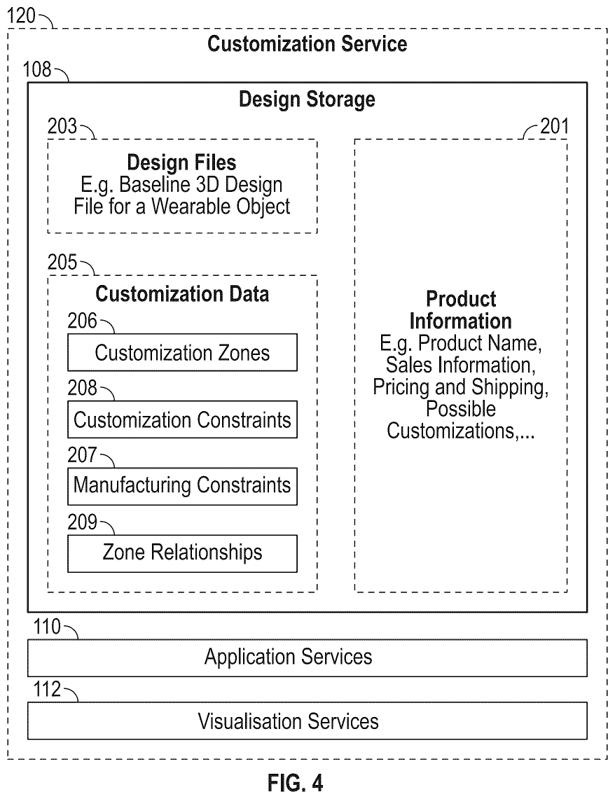

FIG. 4 shows a more detailed view of the design storage 108 which may be part of the customization service 120 of the computing environment 100. The design storage 108 may include stored 3D design files 203 representing a wearable product. The design files 203 may take the form of original raw 3D data such as an STL file, for example. These STL files (or other file format for a 3D design) may be uploaded to the design storage 108 as baseline 3D design files for products identified in product information 201. The design files 203 may be uploaded by the product designer and/or manufacturer. Design storage 108 may also include storage of customization data 205. The customization data 205 may generally be data that defines how each design file 203 can be modified and customized according to the preferences or needs of end-users of the wearable product and the specifications of the designer, and further may take into account manufacturing requirements and functional requirements for the object. For example, there may be separate customization data 205 for each baseline 3D design file.

For example in eyewear applications, the baseline 3D design file for an object may include a baseline model for an eyewear frame. The product information may include general sales information about products available for purchase from manufacturers and/or designers.

In particular, the customization data 205 may define various zones of customization which allow modification of the sizing, spacing, and other dimensions of the wearable product associated with the design. The customization zones 206 may be parts of the object in the design file of which the dimensions can be altered to come to a customized design to fit a particular end-user of the wearable object. The customization data 205 may further include customization constraints 208 defining the dimensional changes that can be made to each of the customization zones of the baseline 3D design without detrimentally impacting the design, such as to a point that it no longer meets the aesthetic an/ord functional requirements, such as defined by the designer.

For example for an eyewear frame, the customization zones 206 may be regions spanning the width of the nose bridge (DBL), the width and height of the lens contour (A-size, B-Size), the length of the side legs (temple length), the tilt angle (pantoscopic angle), the face form angle (FFA), and/or the like. For example, for an eyewear product, the customization constraints may be the maximum deviations from the original proportions of the baseline eyewear frame that ensure the shape of the spectacle frame keeps the aesthetic appearance intended by the designer of the wearable object, the functional requirements generally desired by end-users, and the limitations in terms of manufacturability of the object. Example constraints imposed by a designers could be that the lens width should be a minimum of 40 mm and a maximum of 52 mm, the temple length should be a minimum of 130 mm and a maximum of 180 mm, the width of the nose bridge should ensure the distance between the lenses is a minimum of 10 mm and a maximum of 20 mm, A-size should be a minimum of 50 mm and a maximum of 58 mm, DBL should be a minimum of 13 mm and a maximum of 20 mm, frame face form angle should be between 0-5 degrees extra compared to the base model, and/or the like.

Additionally, the customization data 205 may include manufacturing/printability constraints 207 and zone relationships 209. The manufacturing constraints 207 generally define changes that can be made to a particular design without detrimentally impacting the design to a point that it can no longer be successfully manufactured, for example by 3D printing. In some embodiments, the manufacturing constraints may be defined by the product manufacturer and/or designer as part of the general design process. Alternatively, the manufacturing constraints 207 may be defined by the customization service 120 when the designs 203 are initially stored. The zone relationships 209 generally take the form of a data set that defines relationships between different zones of customization. For example, the zone relationships 209 may be defined so that when a modification is made to one zone defined in the customization data 205, changes are automatically made to other zones in response to that modification data. The zone relationships 209 may be used to provide the ability to make more significant customizations without running afoul of the printability constraints 207 associated with a particular design file 203. Printability constraints 207 differ from customization constraints 208, in that adherence to printability constraints means the modified design can be printed, even if it does not meet functionality or aesthetic requirements, while adherence to customization constraints means the modified designs meet functionality and aesthetic requirements, even if the modified design is not physically able to be manufactured.

For example in eyewear applications, the manufacturing constraints may include limits on the printability of the frame by additive manufacturing techniques ensuring adequate strength of the final product. For example in eyewear applications, the zone relationships may include the necessity to modify the width of the eyewear frame lens contour if the height of the eyewear frame lens contour is modified. Printability constraints may for example apply when prefabricated metal temples are used with a 3D printed frame. The printed frame will have cavities of a predetermined fixed size for insertion of the prefabricated metal temples. To ensure enough strength after printing, sufficient material is needed around the cavities, which imposes a minimum size/width of the frame in the region of the cavities. If the frame size/width would be too small, the region around the fixed size cavities would be too fragile and prone to breaking during manufacturing.

The design storage 108 may include product information 201. The product information 201 may include general sales information about products available for purchase from manufacturers and/or designers. For example, the product information 201 may identify the product by name. The product information 201 may also have an SKU associated with the product, as well as pricing and shipping information. Product information 201 may further include descriptive information regarding the customizations that may be made to the product via the customization service 120, which may provide explanatory information with regard to the customization data 205.

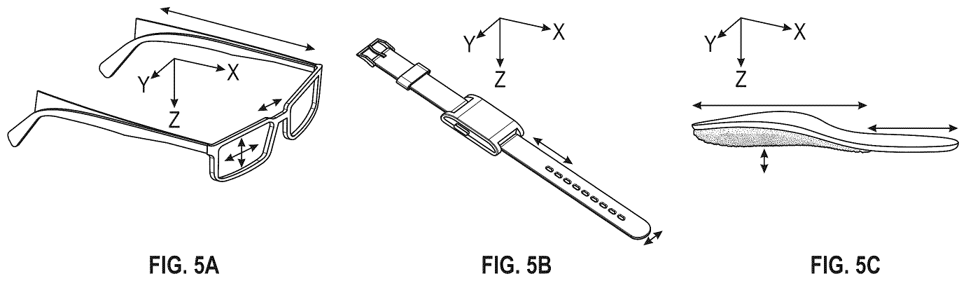

FIG. 5a shows example customizable features for an eyewear product. FIG. 5b shows example customizable features for a wristwatch. FIG. 5c shows example customizable features for an insole. Other examples of customizable products are smart products comprising projected displays. The arrows in FIG. 5a-c indicate possible example geometric variations in possible example customization zones.

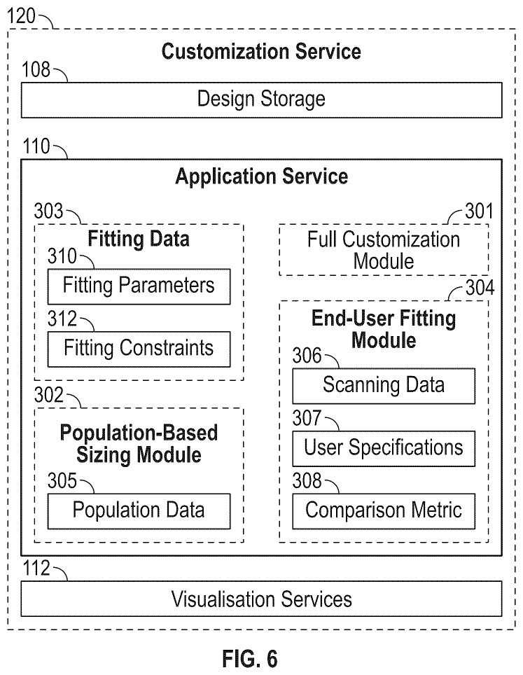

FIG. 6 shows a more detailed view of the application services 110 which may be part of the customization service 120 of the computing environment 100.

The application services 110 may include a full customization module 301 and stored fitting data 303. The full customization module 301 may be configured to customize a baseline 3D design file of a wearable object to a 3D anatomy scan or model of a potential end-user, according to the fitting data 303. The full customization module 301 may be configured to modify the baseline 3D design file for the object by virtually superimposing the baseline 3D design over a 3D anatomy model of a potential end-user.

The full customization module 301 may perform full customization according to the following technique for eyewear applications, or any other suitable technique for any other wearable object known in the art:

A full customization module for constructing custom eyewear products may include:
receiving data indicative of a baseline 3D design for an object, customization data 205 for the object, and/or product information 201 associated with the object;
receiving a 3D model associated with an anatomical feature of an end-user of the object;
receiving fitting data 303;
superimposing the baseline 3D design for the object over the 3D model associated with the anatomical feature of the end-user of the object;
identifying a plurality of visual elements used to modify the baseline design wherein the plurality of visual elements comprise slider elements, and wherein movement of the slider elements is indicative of a change to the physical dimensions of the baseline design;
calculating adjustments of the slider elements for the plurality of visual elements based on the fitting data;
modifying the baseline design within the limits of the received customization data.

The fitting data 303 may be based on historic information or statistical data relating to generalized preferences for users of products of the same type. The fitting data may include fitting parameters 310 and fitting constraints 312. The fitting parameters 310 generally take the form of a data set that defines relationships between the dimensions and position of a wearable object and the anatomy features of a wearer/end-user. The fitting parameters 310 may include positioning parameters that determine the fit, i.e. the functional and aesthetic suitability of a wearable object for a wearer. Functional suitability may include for example, comfort, user experience, manufacturability of auxiliary components of the wearable product, or proper functioning of projections in smart products. Aesthetic suitability may include for example, a generic set of rules on proper fitting, a set of rules relating to the particular shape of the wearable product, a set of rules relating the anatomy shape or a black-box system.

For example for an eyewear frame, the fitting parameters may include: the width of the frame compared to a wearer's face, the position of the frame on a wearer's face, the position of the frame compared to a wearer's pupils, the position of the frame compared to a wearer's eyebrows, the position of the lens that will be inserted in the frame compared to the wearer's pupils, the alignment between a display projected in the lens and the wearer's pupils, and/or the like.

Functional suitability for an eyewear product may include comfort and visual experience for the wearer of an eyewear product. This may relate to lens position, based on average ideal values, based on values for specific lens types (e.g. multifocal lenses), or based on prescription-related factors (e.g. vision impairment corrections may require a threshold lens thickness and a corresponding minimum frame thickness). This may further relate to manufacturability of auxiliary components such as lenses, and to proper display of projections in smart glasses. Aesthetic suitability may be based on a set of rules on generating a fit on a person's face that gives a pleasing look. This may for example be a generic set of rules on proper fitting of an eyewear frame, a set of rules relating to the particular shape of the eyewear frame (e.g. round frames may require a fit closer to the face than rectangular frames), a set of rules relating the facial shape of the wearer (e.g. an elongated facial shape may require a different fit than a rounded facial shape), or a black-box system.

The fitting constraints 312 may generally define changes that can be made to a particular design without detrimentally impacting the successful use of the wearable product by the end-user. The fitting constraints 312 may include ranges of allowable values for the fitting parameters, and may in particular define a suitable (e.g., the optimal) fitting position and a maximum deviation that can occur for each of the fitting parameters for the wearable object compared to the wearer's anatomy. The fitting constraints 312 may be defined by the designer or as part of the customization process and may be based on collective historic data of a range of wearers/end-users.

For example for an eyewear frame, the fitting constraints may be the maximum deviations in three dimensions between parts of the end-user's face and parts of the eyewear frame that ensure an optimal position of the eyewear product on the end-user's face, safeguarding for example a correct position of the lenses for optical functioning of the eyewear product, comfort for the wearer, an aesthetically pleasing fit and size, a correct display of projections in the lenses, and/or the like. Smart glasses, such as virtual reality (VR) glasses, augmented reality (AR) glasses or mixed reality (XR) glasses, may include a projector that generates a display on the lens. The display provides the user with information such as for example navigation, incoming smartphone messages, or entertainment content. Optimal display of the projection requires alignment of the position of the eyewear frame's lenses with the user's eyes.

The application services 110 may further include a population-based sizing module 302 configured to generate virtual 3D anatomy models from actual 3D anatomy models. The population data 305 may include a set of actual 3D anatomy models referred to as a base population including a set of actual 3D anatomy scans of persons representative of different morphologies that may occur in a population of consumers or end-users for which the wearable object is intended. In some embodiments, this base population is the fitting population discussed herein. In some embodiments, the fitting population further includes one or more virtual 3D anatomy models generated from the base population. In certain embodiments, the fitting population may only include virtual 3D anatomy models. In certain aspects, the fitting population includes a number of virtual 3D anatomy models that is larger than the number of 3D anatomy scans in the base population. The fitting population may be configured to represent a broad range of morphologies that may be expected to occur in a population of persons.

For example in eyewear applications, a base population may include a series of 3D facial scans of persons that could reasonably or statistically be expected to exhibit similar facial features. A generated set of virtual 3D anatomy models may include a series of 3D facial models generated based on the one or more of the 3D facials scans of the base population, such as via linear combination, or other interpolations or extrapolations. As an example, a base population of 100 actual 3D anatomy models may be able to generate 1000 virtual 3D anatomy models using different linear combinations of different actual 3D anatomy models. The linear combination of different actual 3D anatomy models may refer to linear combinations of features (e.g., anatomical features, measurements, etc.) of the actual 3D anatomy models.

The virtual 3D anatomy models may be created through random linear combinations of the actual 3D anatomy models of potential wearers of a wearable object included in the base population. This can be done, for example using a statistical shape model. One of the main methods for computing a statistical shape model is principal component analysis (PCA). Using PCA each 3D anatomy model within a set of 3D anatomy models is represented as a linear combination of basic shape segments (for example as a mesh made up of triangles or rectangles) wherein the coefficients of each linear combination follow a normal statistical distribution (i.e. a Gaussian statistical distribution). New 3D anatomy models can be constructed by taking linear combinations of the basic shape segments, where the coefficients are taken randomly from the corresponding normal statistical distributions.

For example for eyewear applications, a statistical shape model may be used to generate virtual 3D anatomy models. The statistical shape model may be constructed by segmenting facial scan images of a potential wearer of an eyewear frame into basic shape segments (for example resulting in a mesh made up of triangles or rectangles). The algorithm may analyze the shapes of a set of facial anatomy models and create a statistical shape model. A virtual 3D facial model can be generated using random linear combinations from the corresponding normal statistical distributions of basic shape segments obtained from the base population of actual 3D facial models.

The population-based sizing module 302 may further be configured to perform an initial batch fitting on the fitting population, and to receive instructions generated by the full customization module 301. The initial batch fitting customizes the baseline 3D design file to each 3D anatomy model of the fitting population through use of the full customization module. The initial batch fitting may result in a set of customized 3D wearable object files comprising modified versions of the baseline 3D design file (and possibly the baseline 3D design file itself), the modified versions being customized to the anatomies represented in the 3D anatomy models of the fitting population according to customization data 205 and fitting data 303. Each customized wearable object file may be represented by a datapoint that represents the conversion of the customized wearable object files into, for example, a set of vectors, a data list, or a point cloud of values.

For example for eyewear applications, wherein a baseline 3D design of an eyewear frame is customized to each of the 3D facial models in a fitting population, the dimensions of each customized frame may be determined. Each customized 3D wearable object corresponding to a customized 3D wearable object file may be a virtual customized eyewear frame that may have a specific width of the nose bridge (DBL), a specific width and height of the lens contour (A-size, B-Size), a specific length of the side legs (temple length), and/or a specific tilt angle (pantoscopic angle). These parameters may be represented in a datapoint. The different datapoints representing a specific customized 3D wearable object corresponding to a customized 3D wearable object file, such as a virtual customized eyewear frame, make up a cloud of datapoints.

Different commonly available mathematical techniques or algorithms may be applied to perform the initial batch fitting. The mathematical technique or algorithm may for example use as input a fitting population comprising a set of files with 3D anatomy models (e.g., directly or converted for example to vectors, a list of data, a point cloud of XYZ values, and/or the like). The mathematical technique or algorithm may for example have as output a set of files comprising 3D design of the wearable object that perfectly fit the entire population, or acceptably fit a large part of the population taking into account customization data 205 and fitting data 303 during customization.

The population-based sizing module 302 may further be configured to generate a set of 3D files storing a discrete set of variants of the baseline 3D design of the wearable object, the discrete set of variants accommodating a portion of the 3D anatomy models of the fitting population when taking into account customization data 205 and fitting data 303. The process of generating the discrete variants of the baseline 3D design of the wearable object may include grouping the datapoints, each datapoint corresponding to a virtual 3D design of the wearable object, into clusters. The clusters may be defined as datapoints that are close to each other according to a given similarity metric, the metric being chosen in such a way that a limited number of clusters accommodates a significant part of the population. Each cluster includes a representative datapoint, which may or may not be one of the datapoints corresponding to a customized 3D design of the wearable object generated based on the fitting population, the representative datapoint representing one discrete variant of the baseline 3D design file. The number of clusters may be defined by the designer, the manufacturer or the retailer/seller. Alternatively, the number of clusters may be the result of an iterative algorithm, for example balancing the number of clusters and the percentage of a population accommodated by the discrete variants defined by the clusters.

For example for applications relating to eyewear products, clusters may be formed of different initial datapoints representing virtual eyewear frames, and in each cluster, a representative virtual eyewear frame is chosen. This may correspond to one of the initial datapoint, or may be a statistically determined datapoint representing the cluster. The representative datapoint may correspond to a specific width of the nose bridge (DBL), a specific width and height of the lens contour (A-size, B-Size), a specific length of the side legs (temple length), and/or a specific tilt angle (pantoscopic angle), and/or the like. Accordingly, a datapoint may have several dimensions, and clustering may occur across several dimensions. This may, for example, for a set of 1000 3D anatomy models with 1000 virtual corresponding customized eyewear frames, result in 10 clusters with 10 discrete variants of the baseline eyewear frame design. These 10 discrete variants may, for example, accommodate 90% of the fitting population.

Clusters in a set of 3D wearable object designs may be created using a clustering algorithm, such as for example but not limited to: K-means clustering, agglomerative clustering, density-based spatial clustering (DBSCAN), or Gaussian Mixture Modelling (GMM). The number of clusters may be pre-defined as input for the clustering algorithm, resulting in a certain percentage of the population that can be accommodated with a wearable object within the limits of the customization and fitting constraints. Alternatively, the required percentage of the population that needs to be accommodated may be pre-defined as input for the clustering algorithm, resulting in a certain number of clusters.

A K-means clustering algorithm may, for example, be used to generate clusters. K-means clustering allows identification and analysis of groups that form organically, rather than pre-defining groups before analysis of the data.

A K-means algorithm is able to find groups in uncategorized data, with the number of groups represented by the variable K. The algorithm iteratively assigns each datapoint within a dataset to one of K groups based on a similarity metric. The algorithm is centroid-based and includes a first step of random initialization wherein K number of centroids are placed randomly, a second step of cluster assignment wherein each datapoint is assigned to a cluster based on the distance to the closest centroid, a third step of centroid optimization wherein centroids are moved to the means of datapoints of the same cluster, and a repetition of the steps of cluster assignment and centroid optimization until convergence is reached. Implementation of a K-means clustering algorithm may require the pre-definition of two parameters: the number of centroids K, and the distance metric. For example, when using quadratic distance as a distance metric, datapoints are grouped based on the lowest sum of the quadratic distance from each datapoint to the centroid of the cluster it is assigned to. Depending on the initialization of centroids, a K-means clustering operation may converge to a local minimum, which may require repeating the clustering operation a number of times with different initializations of centroids.

The K-means clustering algorithm may for example use as input a set of datapoints representing 3D design files for wearable objects customized to the 3D anatomy models of a fitting population, a pre-defined number of clusters K, and a quadratic distance metric. The K-means clustering algorithm may for example have as output a set of K centroids of the K clusters, each centroid of a cluster being a collection of datapoint parameters values which define a discrete variant of the baseline 3D design of the wearable object. Multiple iterations may be undertaken to come to a feasible clustering and a corresponding set of discrete variants of the baseline 3D design of the wearable object, to accommodate a sufficient percentage of the fitting population.

The population-based sizing module 302 may optionally be configured to perform an additional batch fitting on the fitting population, the additional batch fitting verifying for each 3D anatomy model in the fitting population if at least one discrete variant of the baseline 3D design file fits the 3D anatomy model according to the customization data 205 and the fitting data 303. The initial batch fitting may result in the determination of the percentage of the fitting population that can be accommodated with the discrete variants resulting from the initial batch fitting. Several iterations of batch fittings and clustering may be performed.

For every 3D baseline design of a wearable product, such as an eyewear frame, every person is considered to have an optimal size fit within certain tolerances (style preferences such as e.g. preferring oversized glasses are not taken into consideration). The optimal size fit and tolerances are determined based on historic data of a group of users of similar types of wearable products. For example an eyewear frame with an optimal nose width for a particular wearer of 2 cm may still be accommodated with an acceptable nose width of up to 0.5 mm narrower than the optimal nose width and up to 1.5 mm wider than the optimal nose width. The clustering algorithm may be built to minimize the deviation from the optimal size for every person in the population.

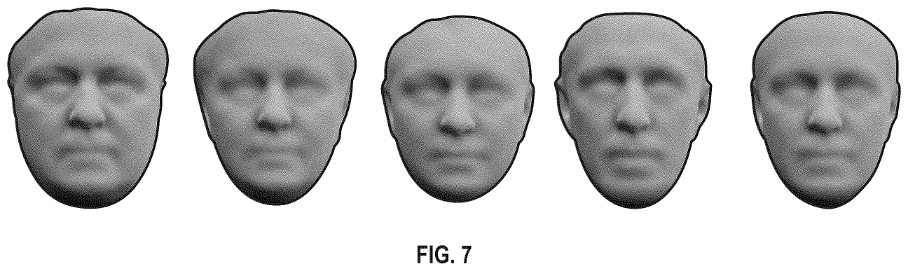

FIG. 7 shows examples of virtual 3D facial models generated using a statistical shape model created through random linear combinations of a set of actual 3D anatomy models of potential wearers of a wearable object included in a base population.

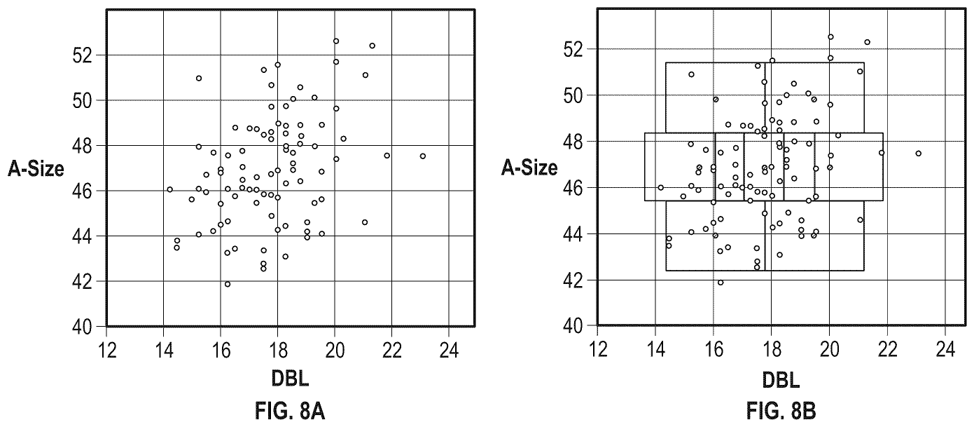

FIGS. 8a-b show an example of clustering in an eyewear application. The applied clustering algorithm takes into account two fitting parameters, in this case A-size and DBL, and therefore there are only two dimensions. However, additional and/or alternative fitting parameters and accordingly additional and/or alternative dimensions may be used for the clustering. FIG. 8a shows datapoints representing eyewear frame designs customized to a 3D anatomy model, each datapoint corresponding to a specific combination of A-size and DBL. FIG. 8b shows clustering of the datapoints, in which 95% of the datapoints in the dataset are accommodated by 7 clusters. Each cluster has its representative datapoint at its center, the representative datapoint corresponding to a specific combination of A-size and DBL, and representing a discrete variant of the baseline eyewear frame design. There is overlap in some regions of some of the clusters. This is where more than one accommodating discrete variant of the baseline eyewear frame design is available for the virtual end-user represented by the corresponding datapoints.

For example for applications relating to eyewear products, the designer and manufacturer may jointly choose the number of clusters and the minimum % of the population that needs to be accommodated. Several iterations of batch fittings and clustering may be performed to come to a satisfactory result.

The application services 110 may also include an end-user fitting module 304.

The end-user fitting module 304 may include stored 3D scanning data 306 associated with a user and stored user specifications 307. The 3D scanning data 306 and the user specifications 307 may be uploaded by the end-user, or alternatively by the product retailer/seller. Scanning data 306 may include information associated with 3D scans of anatomy parts associated with the end-user. The 3D scans may be made using a scanning device 116. The user specifications 307 may include data which allows the system to better fit products to the end-user's individual needs, including for example physical attributes, lifestyle characteristics, comfort preferences and/or medical conditions.

For example in eyewear applications, the 3D scanning data associated with the end-user may be a 3D facial scan of the user, which may be created using any type of camera system. The scanning system may include means for making measurements to allow lens centration, for example through virtual centration. Virtual centration identifies the wearer's pupil locations on the 3D facial scan. When the 3D model of the frame is superposed on the 3D facial scan, the virtual centration measurements allow position of the lenses in the eyewear frame so the pupils and lenses are aligned for optimal functioning of the eyeglasses.

For example in eyewear applications, the user specifications may include information relating to the needs and preferences of the end-user relating to lifestyle, lenses, wear comfort and/or visualization of projected displays. Information relating to lifestyle may include, for example the intended use of the eyewear product such as for reading, watching TV, office work, sports, and/or the like. Information relating to lenses may include data on the end-user's prescription for vision correction, lens centration, preferences on setting of a lens position that is optimized for the wearer, for example in case of prescription eyewear to correct optical deficiencies, and/or the like. Prescription data may include data or measurements for bifocal, trifocal, or multifocal lenses. Lens parameters may further include information on previous glasses, lens type, and pupillary distance (PD). Lens parameters may further include lens offset (x & z), pantoscopic angle (PA), corneal vertex distance (CVD), lens face form angle (LFFA), minimal eye point height (EPH), minimal B-size, minimal distance to upper, minimum far zone and minimum corridor length, and/or the like. The values for the lens parameters may be selected from an ideal value and a range of tolerated values for the lens parameters. Information relating to wear comfort may include position on the nose and cars and lens centration. Information relating to visualization of projected displays may include digital display parameters for the end-user, wherein the digital display parameters ensure display alignment providing optimal viewing performance, for example in case of smart eyewear, AR, VR or XR glasses. Digital display parameters may include information that allows for prescription lens integration to be taken into account. Digital display parameters may include information that allows positioning of the optical components in front of the eyes for optimal visibility of the displays, with each pupil aligned within the eyebox. Example digital display parameters may include pupillary distance, segment height, and/or eyepoint relief. The user specifications for eyewear products may further include information on personal preferences of the end-user, such as for example but not limited to preferences on the fit of the eyewear frame, preferences on the visualization type of projected displays.

The end-user fitting module 304 may be configured to fit the baseline 3D design file to the 3D scanning data 307 associated with the user, and to receive instructions generated by the full customization module 301. The full customization module 301 may be configured to customize the baseline 3D design file to the 3D anatomy scan of the end-user, taking into account customization data 205 and fitting data 303, and further taking into account user specifications 307, resulting in an end-user customized 3D design file. The end-user fitting module 304 may further be configured to store the end-user customized 3D design file. The end-user fitting module 304 may further include a comparison metric 308 and may further be configured to allocate a fit score to define the overlap of a given 3D design file to a reference 3D design file. The comparison metric 308 may be based on deviations of the dimensions and/or shape of part of the wearable object in a 3D design file compared to a reference object in a reference 3D design file. The part of the wearable object considered in the comparison metric may correspond to the customization zones 206. A fit score may be allocated based on the overlap between each discrete variant and the customized modified design, with a higher degree of deviation of the comparison metric resulting in a lower fit score for the discrete variant that is being scored. The allocation of the fit score may take into account the fitting data 303 and the user specifications 307.

For example in eyewear applications, a simple fit score could be a function that equals the sum of one or more of the following components:
squared difference between DBL of the discrete variant and DBL of the customized modified design,
squared difference between lens width of the discrete variant and lens width of the customized modified design,
squared difference between lens height of the discrete variant and lens height of the customized modified design,
squared difference between the full width of the frame of the discrete variant and the full width of the frame of the customized modified design.

Alternative functions may for example include assigning weights to the different components to make certain measurements more important than others.

For example in eyewear applications, the end-user customized 3D design file may include a fully customized eyewear frame for the end-user, generated by modification of the baseline 3D design for the eyewear frame to fit the 3D facial scan of the end-user. The full modification may take into account customization data aimed at ensuring the eyewear frame is not modified beyond pre-defined functional and aesthetic constraints. The full modification may further take into account fitting parameters, aimed at ensuring the eyewear frame is modified in such a way that it fits the facial scan within pre-defined functional and aesthetic size and user preferences based on historic and statistical data. The full modification may further take into account user requirements, aimed at ensuring the eyewear frame meets the needs of the end-user in terms of lifestyle, lens requirements, comfort and visualization of projected displays. If there is no fully customized fit possible within the limitations of the customization parameters and the fitting parameters, the end-user may be required to choose a different baseline design. In eyewear applications, a comparison metric for an eyewear frame may be based on the overlap of the dimensions of the nose bridge (DBL), the width and height of the lens contour (A-size, B-Size), the length of the side legs (temple length), and/or the tilt angle (pantoscopic angle). A number of discrete variants of a baseline 3D design for an eyewear frame may be compared to an end-user customized 3D design file comprising a fully customized eyewear frame personalized to fit the facial scan of the end-user.

Alternatively, the end-user fitting module 304 may be configured to superimpose the discrete variant files directly onto the 3D scanning data 307 associated with the user. In this alternative method, a fit score may be allocated to determine the fit of a given 3D design file to the 3D scan associated with the user. In this alternative method, the comparison metric 308 may take into account fitting data 303 and user specifications 307. A fit score may be allocated based on the degree to which a variant frame meets the fitting constraints and corresponds to the user specifications, with a higher degree of deviation of the comparison metric resulting in a lower fit score for the discrete variant that is being scored. For example, a fit score can be constructed as the sum of different components, where each component corresponds with the weighted squared difference between two dimensions.

A fit score may be allocated to each discrete variant. The comparison metric and fit score allocation may for example be based on a distance function based on a least squares approach. The fit score may take into account the fitting data to ensure a higher score is allocated if the discrete variant better meets the pre-defined functional and aesthetic size and user preferences based on historic and statistical data. The fit score may further take into account the user preferences to ensure a higher score is allocated if the discrete variant better meets the needs of the end-user in terms of lifestyle, lens requirements, comfort and/or visualization of projected displays.

A fit score could also be steered by approximation of ideal virtual centration values in case of prescription eyewear or ideal eyebox position in case of smart eyewear. Common virtual centration values, which determine the position of the pupil relative to the lens, include interpupillary distance, eyepoint height, pantoscopic angle, frame face form angle and cornea vertex distance. The system may select among the plurality of discrete variants for a baseline eyewear frame, a frame that has a suitable (e.g., threshold, the highest, etc.) fit score based on comparison of the fit of the discrete variants to the wearer of an eyewear frame. There may be more than one frame with the same fit score, which may allow the end-user to choose between both frames. If based on the comparison metric, there is no fit within the limitations of the fitting parameters and/or the user specifications, there may be no selection of an eyewear frame for the user in question, and the end-user may be required to choose a different baseline design. This may for example occur, if none of the discrete variants of the baseline design is able to accommodate the required lenses for the user.

The end-user fitting module 304 may further be configured to select the discrete variation of the 3D baseline design with a suitable (e.g., the highest, above a threshold, etc.) fit score for the individual end-user of the wearable object.

The user specifications 307 may further include individual design customization preferences. The individual design customization preferences may generally include data associated with past 3D design modifications made in relation to the end-user with respect to a manufactured object. The individual design customization preferences may be generally provided to allow an end-user registered in the system to reuse design customizations, or otherwise use them to better specify future customizations for similar types of products. The end-user fitting module 304 may further be used to allow individual end-users to manage their designs and preferences according to one or more embodiments. In some embodiments end-users are able to customize 3D designs, including for example color, material and/or hinge type, to suit for example aesthetic tastes and functional needs.

The end-user fitting module 304 may be configured to operate without generating a graphical user interface, or any other form of display of the 3D anatomy scanning data associated with the user, the baseline 3D design file of the wearable object, the end-user customized 3D design file, the discrete variants, or any combination thereof. Alternatively, the end-user fitting module 304 may be configured to generate some type of visualization or display, for example, of the selected discrete variant superimposed over the 3D anatomy scan of the end-user. The visualizations or displays may be generated using the visualization services 112.

For example in eyewear applications, a visual display may be generated visualizing selected discrete variant(s) of the eyewear product superimposed over the 3D facial of the end-user.

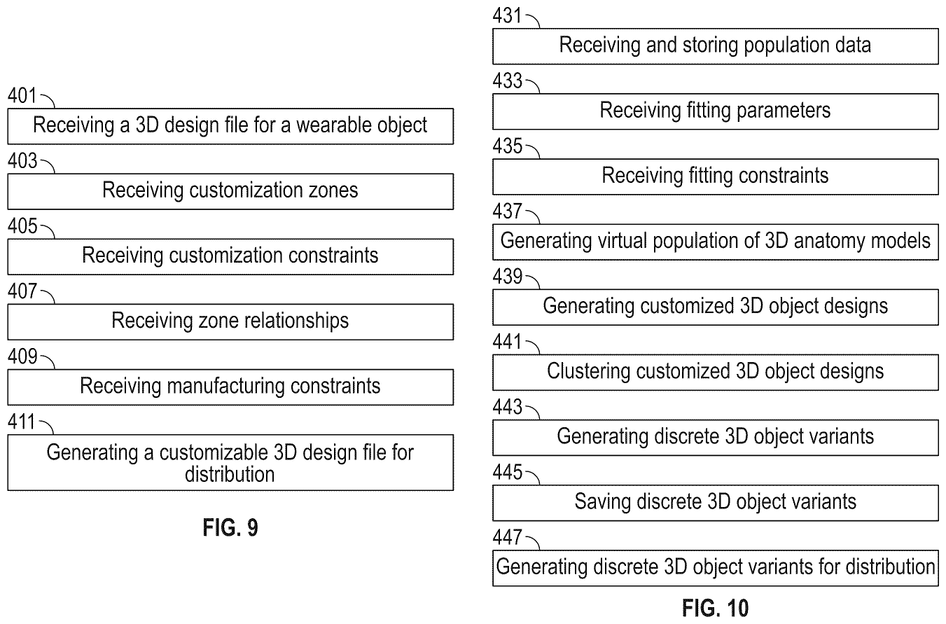

FIG. 9 shows a flow chart detailing an example high-level process by which a computer network 102 may receive input from a designer, manufacturer or retailer producing a customizable 3D design. The process begins at block 401, where the computer network 102 receives a file associated with a wearable object design, for example from a designer, manufacturer or retailer, for example an STL file. Next, the process moves to block 403. There, the computer network 102 may receive zones of permitted customizations, for example defined by the designer, manufacturer or retailer. Generally, the zones of permitted customizations may be defined by the designer/manufacturer to allow customizations which fit general user preferences with regard to aesthetics, comfort and functional needs, but do not change the key design and branding features associated with the object.

For example in eyewear applications, a designer of eyeglasses may produce a design which has lenses of a certain general shape. Because this shape is important to the stylistic aspects of that particular eyeglass model, the designer may decide to avoid making the shape of the lens a zone of customization. However, in order to ensure that the eyeglasses can be made to fit the customer in the best possible way, a designer may define zones of customization which allow the eyeglasses to be modified to better fit physical characteristics associated with the particular person for whom the eyeglasses are manufactured. For example, a designer may create a design in which the lenses have a square shape. If the customized fit suggests to change the width of the lenses by 20% and the height of the lenses by 10%, the resulting frame will not have square lenses. The designer could thus impose a constraint to keep the lenses square. In this case the customized fit will have to comply with the constraint and the lens will be made for example 15% wider and higher, instead of the optimal 20% width increase and 10% height increase.

The process next moves to block 405, where the computer network 102 may receive customization constraints for the design. These constraints are typically based on the intended aesthetic and overall look of the product as originally intended by the designer. The process may then move to block 407, where the computer network 102 may receive zone relationships. Zone relationships may be defined such that changes to one zone of customization may cause corresponding and/or related changes to a different zone of customization. The zone relationships may be defined such that these related changes automatically take place when certain triggering changes are made in a particular zone. Alternatively, the zone relationships may be defined so that the user is offered an optional corresponding change based on a defined zone relationship. The process next moves to block 409, where the computer network 102 may receive manufacturing constraints on the customization of a design. These constraints are typically based on the manufacturability of the modified design. For example, the constraints may be defined such that a modification (or combination of modifications) may be disallowed because the subsequently manufactured object will possess inherent structural deficiencies. In addition, certain modifications may be constrained because they result in a modified design that cannot be efficiently manufactured, for example using the manufacturing services 106 which are associated with the manufacturing of that particular device.

The process moves to block 411 where the computer network 102 makes the customizable 3D design file available for distribution. The modified design file may be then transmitted to the design storage 108 within the customization service 120. There, the design can be made available for customization, including customization to a particular end-user, or customization to virtual end-users in a fitting population allowing clustering and determination of discrete variants.

FIG. 10 shows a flow chart providing an example of a process by which a set of discrete variants of a product can be generated. The process begins at block 431, where the computer network 102 receives and stores baseline population data, which may for example be uploaded by a retailer or manufacturer. As detailed above, the population data may include a set of 3D anatomy models representing a population of persons statistically expected to have similar anatomy features. The process may then move to block 433, where the computer network 102 may receive fitting parameters that are relevant to ensure an optimal fit of the product on an end-user's anatomy, which may be defined by the designer, manufacturer or retailer. Next, the process moves to block 435. There, the computer system 102 may receive constraints for the fitting parameters, for example defined by the designer, manufacturer or retailer. These fitting constraints are typically ranges and/or optimal values for the fitting parameters that define an acceptable and/or optimal position of the product on the anatomy of the end-user and may be based on statistical or historical user preferences with regard to aesthetics, comfort and functional needs.

Optionally, the process may then move to block 437, where one or more virtual 3D anatomy models are generated, such as using the techniques discussed herein. Accordingly, a fitting population is defined including one or more actual 3D anatomy models and/or one or more virtual 3D anatomy models. The process may then move to block 439. Here, the product may be customized to each of the 3D anatomy models of the fitting population, taking into account the customization constraints, zone relationships and manufacturing constraints, and fitting constraints. This results in a set of customized 3D object designs. The process may then move to block 441. Here the customized 3D object designs may be clustered into a number of groups, and in each group a representative customized 3D object design may be defined. As described above, clustering of the customized 3D object of the fitting population may for example be done using a clustering algorithm such as K-means, in which the number of clusters is pre-defined, and the customized 3D object designs are allocated based on a distance metric such as a quadratic distance.

The process may then move to block 443, where a discrete set of 3D object variants is generated. Each of the representative customized 3D object designs in the different clusters may correspond to a discrete 3D object variant.

Once the discrete set of 3D object variants have been defined, the process moves to block 445 where each of the discrete set of 3D object variants may be saved, for example in the form of an STL file, and to block 447 where a 3D object discrete variant design file is generated for distribution. The 3D object discrete variant design file may be then transmitted to the end-user fitting module 304 within the customization service 120. There, the design can be made available for comparison to an end-user customized object design, allowing selection of the most suitable discrete variant design for the end-user

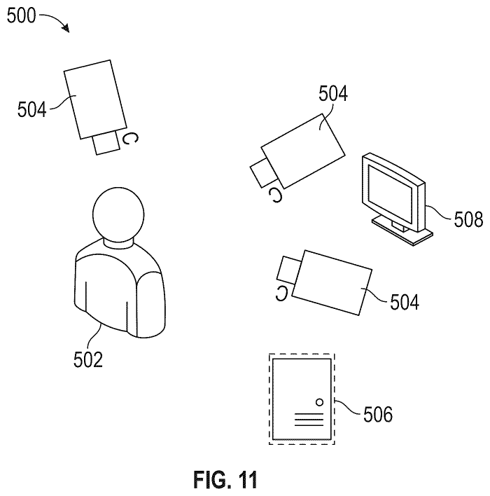

FIG. 11 shows an example of a 3D scanning environment 500 in which a 3D physical scan of an end-user user 502 may be obtained for use in connection with the customization service 120 allowing the system to analyze which discrete variant best fits the physical characteristics of the end-user. In some embodiments, a scanning device 116 may be used to scan the relevant body parts of the consumer. As shown, the 3D scanning environment 500 includes a scanning device having various different components. The components may include a series of cameras 504 attached to movable arms. The cameras in the movable arms may be controlled by controlling software and/or hardware 506 which may be provided by a standard personal computer, or alternatively by specialized scanning controller device. The environment may also include a video monitor 508. The video monitor 508 may be used as an input output device which allows for user interaction. For example, in some embodiments the video monitor may be a touch screen which allows the user to input commands and otherwise control the operation of the 3D scanning environment 500. Using this 3D scanning environment 500, 3D scans of a selected anatomy part of the user 502 may be obtained, and then saved to a computer memory. For example, an end-user 502 may have their face and head scanned by a 3D scanning environment 500. Once the scan has been completed, the end-user fitting module 304 within the application services 110 of the customization service 120 may receive the 3D scan from the user. There, the system can use the 3D scan as part of the fitting, comparison and scoring process for selecting the optimal discrete variant for the user.

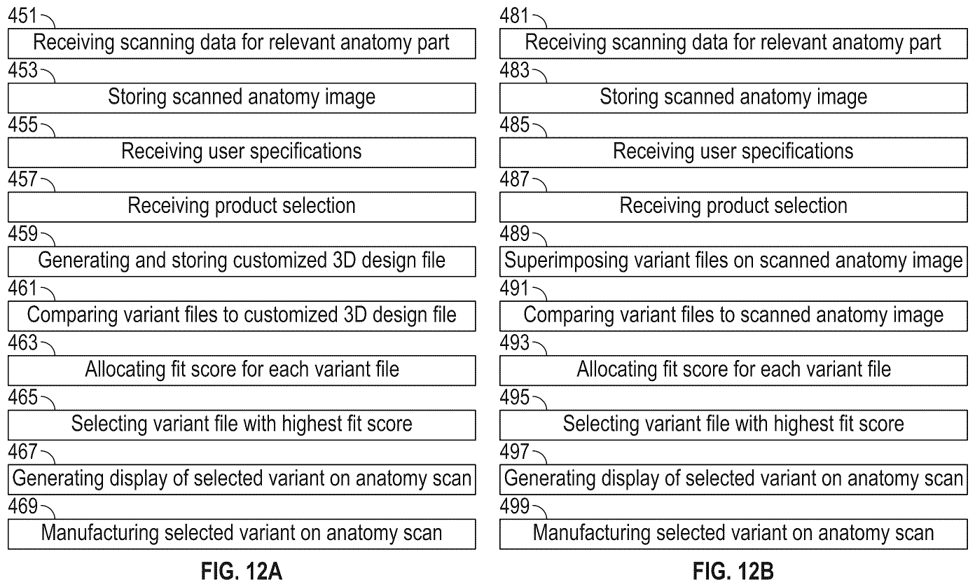

FIGS. 12a-b show flow charts providing example processes by which the customization service can be accessed by an end-user for the selection of a variant that best fits the end-user's anatomy using the end-user fitting module 304.

FIG. 12a shows a flow chart providing an example of a process by which a variant of a 3D product design is selected for a user in accordance with one or more embodiments disclosed herein. In certain embodiments, to allocate a fit score, the set of discrete variants is compared to a fully customized wearable object for the end-user. The process begins at block 451, where the computer network 102 receives a 3D scan of the relevant anatomy part of an end-user, which may be uploaded by the end-user. Next, at block 453, the computer network 102 stores the 3D scan to the scanning data 306 within the end-user fitting module 304 of the application services 110. The process next moves to block 455, where the computer network 102 receives the user specifications 307, which may be uploaded by the end-user. The process next moves to block 457. There, the computer network 102 receives the selection of a product from the design storage 108, selected by the end-user. Once the product has been selected, the process moves to block 459 where a fully customized 3D design of the selected product is created and stored. As discussed in detail above, the customizations may be based on zones of customization constrained by various factors such as printability, design aesthetics, and the like. In addition, in some embodiments, modifications to certain zones of customizability may automatically cause changes to other areas of the baseline design. The customizations may further be based on fitting parameters 310 and fitting constraints 312 ensuring functional and aesthetic position of the product on the anatomy of the end-user. The customizations may further be based on user specifications provided by the end-user ensuring personal preferences and requirements are taken into account. The process then moves to block 461. There, the stored customized 3D design file is compared to the discrete variants of the product generated as detailed above. In some embodiments, the process then moves to block 463, where a fit score is allocated to each variant based on a comparison metric. As discussed in detail above, the comparison metric may take into account overlap/deviation of different zones of the fully customized 3D design and the discrete variants. The process moves to block 465, where a variant is selected, such as a variant having a highest fit score, a suitable threshold fit score, and/or the like. The process then moves to block 467, where a display is generated of the relevant portion of the 3D scan uploaded by the end-user with the selected variant of the product superimposed over the scanned anatomy part. The 3D file of the selected variant may then be sent to a manufacturing service. Once the customized design has been received at the manufacturing service, the process then moves to block 469 where the customized design is manufactured, example using the manufacturing services 106, or obtained from stock.

FIG. 12b shows a flow chart providing an example of an alternative process by which a variant of a 3D product design is selected for a user in accordance with one or more embodiments disclosed herein. In certain embodiments, to allocate a fit score, the set of discrete variants is superimposed on the 3D anatomy scan of the end-user. The process begins at block 481, where the computer network 102 receives a 3D scan of the relevant anatomy part of an end-user, which may be uploaded by the end-user. Next, at block 483, the computer network 102 stores the 3D scan to the scanning data 306 within the end-user fitting module 304 of the application services 110. The process next moves to block 485, where the computer network 102 receives the user specifications 307, which may be uploaded by the end-user. The process next moves to block 487. There, the computer network 102 receives the selection of a product from the design storage 108, selected by the end-user. Once the product has been selected, the process moves to block 489 the variant files are superimposed on the scanned anatomy image of the end-user. The process then moves to block 491 where the variant files are compared to the 3D anatomy scan of the end-user taking into account fitting parameters 310 and fitting constraints 312. Optionally, the process then moves to block 493, where a fit score is allocated to each variant based on a comparison metric. The comparison metric may take into account how the variant compares to the fitting data 303. The process moves to block 495, where a variant, such as with the highest fit, suitable threshold score, and/or the like, is selected. The process then moves to block 497, where a display is generated of the relevant portion of the 3D scan uploaded by the end-user with the selected variant of the product superimposed over the scanned anatomy part. The 3D file of the selected variant may then be sent to a manufacturing service. Once the customized design has been received at the manufacturing service, the process then moves to block 499 where the customized design is manufactured, example using the manufacturing services 106, or obtained from stock.

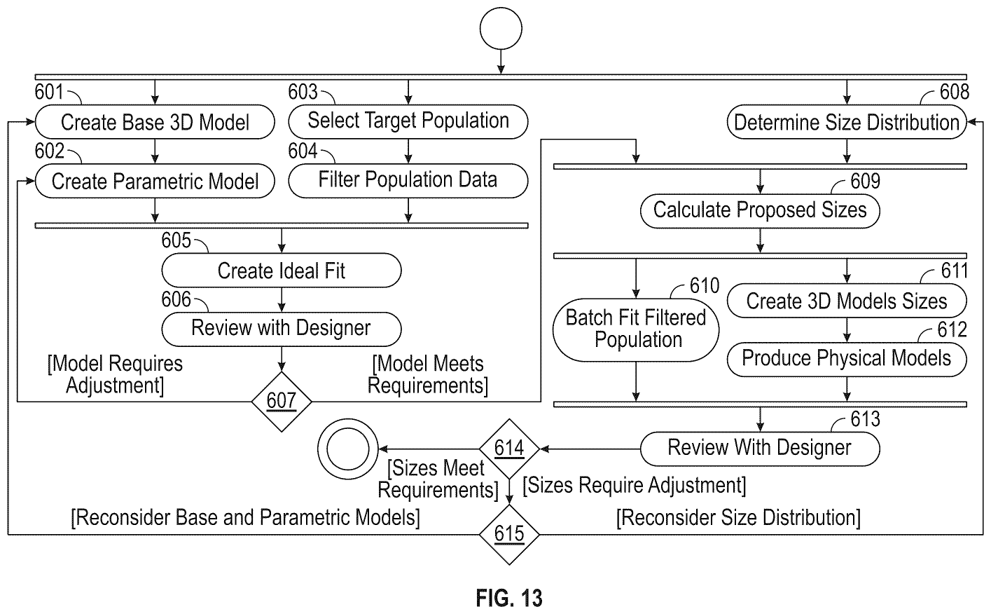

FIG. 13 is a flow chart showing a process where variants of a 3D product design are created in accordance with one or more embodiments disclosed herein.

At 601, a baseline 3D design file for a baseline 3D object is created. The baseline 3D design file defines a baseline 3D model for the object. In an example, the baseline 3D object is eyewear and comprises portions of the eyewear that can be modified, such as one or more of front, temples, etc. In certain aspects, the portions of the eyewear further include nosepads, temple sleeves, and/or the like.

From the baseline 3D design file, a parametric model is created at 602. This parametric model may include the baseline 3D design file as well as customization data as discussed herein. For example, the baseline 3D design file is augmented, such as parameterized, to include extra information, such as in a proprietary file format, such as to adjust the behavior of a rescaling and fitting algorithms on the eyewear.

Optionally, a target population is selected at 603. The target population may define a subset of a population, such as male, female, both male and female, nonbinary, one or more ethnicities, a certain distribution, and/or the like. A fitting population may be generated at 604, such as based on the target population. For example, one or more actual 3D anatomy models and/or virtual 3D anatomy models corresponding to potential wearers within the target population may be used for the fitting population.

At 605, each member (e.g., 3D anatomy model) of the fitting population is fitted with an ideal customization of the parametric model (e.g., a modified 3D design of a customized wearable object, such as rescaling of a baseline 3D design of a customized wearable object). The fitting may take into consideration different constraints discussed herein, such as fitting data, customization data, user preferences, and/or the like, such as aesthetics of the fitted frame, position in front of pupils for optical performance, etc.

At 606 the ideal customizations of the parametric model are reviewed, such as by a designer, such as to ensure the behavior of the fitting and/or resizing is suitable (e.g., ideal). For example, certain designs may be intended to be oversized, or very small. There may also be a limit to how far certain designs can be resized in certain dimensions. For example, a lower limit on nose bridge size may need to be imposed. If approval of the ideal customizations of the parametric model is given at 607, the process continues. If instead at 607, the ideal customizations of the parametric model are determined to need adjustment, the process returns to step 602 to recreate the parametric model, create ideal fits at 605, and review again at 606.

At 608, a size distribution is determined. In certain aspects, this includes both how many discrete variants will be created, and how the feature space will be divided/distributed, such as across a size spectrum. Features used for the size distribution may include one or more of lens size, nose bridge width, lens thickness, and/or the like. For example, nine discrete variants of eyewear may represent combinations of three distinct lens sizes and nose bridge widths. In another example, the distribution may densely cover the center of the target population to provide increased choice for more customers. Alternatively, the distribution may be selected to more sparsely cover the target population, increasing the population with an acceptable or good fit, at the expense of closeness of fit overall. In some cases, a fixed size distribution may be statistically aligned with the results of fitting the fitting population with the parametric model. In some cases, further optimization of the distribution may be performed using clustering algorithms.

At 609, the collection of ideal fits for the population from 605 and the intended size distribution from 608 are used to calculate proposed sizes/modified 3D designs for the discrete variants. These proposed sizes are fitted (e.g., virtually placed) to the fitting population at 610. The proposed sizes can be exported to 3D models in 611, from which physical models can be manufactured at 612.

At 613, the designer reviews the fits, the 3D models, and/or the physical models if created. If at 614, the designer decides the proposed discrete variants meet requirements, the proposed sizes are accepted, finishing the process. If instead the sizes do not meet requirements, the designer decides at 615 to either reconsider the size distribution at 608, or reconsider the base 3D model and parametric model at 601.

In certain cases, the end-user may run through the processes described above online, and place an online order for the wearable object.

Machine learning techniques may be used in combination with any of the above processes. Statistical shape models are only one example of statistical models. Various other techniques in the field of artificial intelligence, machine learning, supervised learning, unsupervised learning, reinforcement learning, self learning, feature learning, anomaly detection, deep learning and the like are known in the art that can be used to perform the tasks described above, such as supplementing (incomplete) user-specific data, analyzing the shape of a user's body part, creating 2D or 3D models or analyzing 2D or 3D image data.

It is to be understood that any feature described in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the disclosure.

1. A method for creating a set of 3D design files for a discrete set of variants of a customizable wearable object, comprising:
receiving a baseline 3D design file for a baseline 3D customizable wearable object;
obtaining a fitting population, comprising a set of 3D anatomy models derived from one or more 3D anatomical scans of one or more individuals, representing potential wearers of the customizable wearable object;
generating a plurality of models of modified 3D customizable wearable objects based on fitting a model of the baseline 3D customizable wearable object to each 3D anatomy model of the set of 3D anatomy models;
selecting one or more of the plurality of models of modified 3D customizable wearable objects as corresponding to the discrete set of variants of the customizable wearable object; and
generating a set of 3D design files for the discrete set of variants of the customizable wearable object for use for manufacturing.

2. The method of claim 1, wherein obtaining the fitting population comprises:
adding a second set of 3D anatomy models set (3D anatomy models, wherein the second set of 3D anatomy mode generated from linear combinations of the set of 3D anatomy models.

3. The method of claim 1, wherein obtaining the fitting population comprises:
adding a second set of 3D anatomy models to the set of 3D anatomy models, wherein the second set of 3D anatomy models is generated from a statistical shape model based on the set of 3D anatomy model.

4-7. (canceled)

8. The method of claim 1, wherein generating the plurality of models of modified 3D customizable wearable objects comprises:
virtually placing the model of the baseline 3D customizable wearable object one each 3D anatomy model of the set of 3D anatomy models.

9. The method of claim 1, further comprising receiving customization data for the baseline 3D customizable wearable object, the customization data including one or more limitations on changes to one or more spatial parameters of the baseline 3D customizable wearable object, and wherein generating the plurality of models of modified 3D customizable wearable objects comprises:
modifying the model of the baseline 3D customizable wearable object within the one or more limitations.

10. The method of claim 1, further comprising receiving fitting data comprising at least one of ranges or values for dimensions of parts of the baseline 3D customizable wearable object suitable for the set of 3D anatomy models, and wherein generating the plurality of models of modified 3D customizable wearable objects comprises:
modifying the model of the baseline 3D customizable wearable object based on the fitting data.

11. The method of claim 1, wherein selecting the one or more of the plurality of models of modified 3D customizable wearable objects comprises:
creating a set of datapoints corresponding to the plurality of models of modified 3D customizable wearable objects;
grouping the datapoints into clusters based on dimensions of the plurality of models of modified 3D customizable wearable objects; and
determining, for each cluster, a representative datapoint corresponding to a variant of the discrete set of variants of the customizable wearable object.

12. The method of claim 1, wherein the baseline 3D customizable wearable object comprises an eyewear product.

13. The method of claim 1, wherein the baseline 3D customizable wearable object comprises a smart eyewear product.

14. The method of claim 1, further comprising:
receiving a first 3D anatomy model comprising a first 3D anatomy scan of at least a part of an anatomy of a first user;
generating a first model of modified 3D customizable wearable object based on fitting the model of the baseline 3D customizable wearable object to the first 3D anatomy model;
comparing the discrete set of variants to the first model of modified 3D customizable wearable object according to a comparison metric to generate fit scores for the discrete set of variants; and
selecting one or more variants of the discrete set of variants based on the fit scores.

15. The method of claim 14, further comprising receiving user specifications comprising one or more preferences or one or more requirements of the first user relating to the baseline 3D customizable wearable object, wherein generating the first model of modified 3D customizable wearable object is based on the user specifications.

16. The method of claim 14, further comprising displaying an image superimposing a depiction of the one or more variants on the first 3D anatomy model.

17. The method of claim 1, further comprising:
receiving a first 3D anatomy model comprising a first 3D anatomy scan of at least a part of an anatomy of a first user;
virtually placing models of the discrete set of variants on the first 3D anatomy model to generate fit scores for the discrete set of variants based on a comparison metric; and
selecting one or more variants of the discrete set of variants based on the fit scores.

18. The method of claim 17, further comprising receiving user specifications comprising one or more preferences or one or more requirements of the first user relating to the discrete set of variants, wherein the comparison metric is based on the user specifications.

19. The method of claim 17, further comprising displaying an image superimposing the models of the one or more variants on the first 3D anatomy model.

20-32. (canceled)

33. A non-transitory computer readable medium storing instructions, which when executed by a computing system, cause the computing system to perform operations for creating a set of 3D design files for a discrete set of variants of a customizable wearable object, the operations comprising:
receiving a baseline 3D design file for a baseline 3D customizable wearable object,
obtaining a fitting population comprising a set of 3D anatomy models derived from one or more 3D anatomical scans of one or more individuals, representing potential wearers of the customizable wearable object.
generating a plurality of models of modified 3D customizable wearable objects based on fitting a model of the baseline 3D customizable wearable object to each 3D anatomy model of the set of 3D anatomy models;
selecting one or more of the plurality of models of modified 3D customizable wearable objects as corresponding to the discrete set of variants of the customizable wearable object, and
generating a set of 3D design files for the discrete set of variants of the customizable wearable object for use for manufacturing.

34. A computing system comprising one or more memories and one or more processors configured to:
receive a baseline 3D design file for a baseline 3D customizable wearable object;
obtain a fitting population comprising a set of 3D anatomy models derived from one or more 3D anatomical scans of one or more individuals, representing potential wearers of a customizable wearable object;
generate a plurality of models of modified 3D customizable wearable objects based on fitting a model of the baseline 3D customizable wearable object to each 3D anatomy model of the set of 3D anatomy models;
select one or more of the plurality of models of modified 3D customizable wearable objects as corresponding to a discrete set of variants of the customizable wearable object; and
generate a set of 3D design files for the discrete set of variants of the customizable wearable object for use for manufacturing.

35. (canceled)
